(19) United States
(12) Patent Application Publication
(10) Pub. No.: US 2008/0188921 A1
(43) Pub. Date: Aug. 7, 2008

(54) Prosthesis Deployment Apparatus and Methods

(75) Inventors: Dwayne S. Yamasaki, Rohnert Park, CA (US); Kevin Wu, Cambridge, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/670,468

(22) Filed: Feb. 2, 2007

(51) Int. Cl. A61F 2/06

(52) U.S. Cl. 623/1.13; 623/1.15

(57) ABSTRACT

A signal lead for an electromagnetic localization marker, which is coupled to a prosthesis, is releasably coupled to the prosthesis. In one embodiment, the lead and electromagnetic localization marker are both releasably attached to the prosthesis through a coupling. In another embodiment, a portion of the lead adjacent to the electromagnetic localization marker is electrolytically dissolved in vivo and the lead removed.

### FIELD OF THE INVENTION

The invention relates to prosthesis deployment and more particularly to prosthesis having electromagnetic markers with leads that are releasably coupled to the prosthesis.

### BACKGROUND OF THE INVENTION

Tubular prostheses such as stents, grafts, and stent-grafts (e.g., stents having an inner and/or outer covering comprising graft material and which may be referred to as covered stents) have been widely used in treating abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier. The graft material for any of the prostheses described herein also can be any suitable material such as Dacron® material or expanded polytetrafluoroethylene (ePTFE).

Aneurysms generally involve abnormal widening of a duct or canal such as a blood vessel and generally appear in the form of a sac formed by the abnormal dilation of the duct or vessel. The abnormally dilated vessel has a wall that typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft spans across and extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened, and a graft is sutured into position bypassing the aneurysm. The endovascular approach, which has been used to deliver stents, grafts, and stent grafts, generally involves cutting through the skin to access a lumen of the vasculature. Alternatively, lumenar or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly to a portion where it spans across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter and self expands upon retraction or removal of the sheath at the target site. More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to distal end of the inner tube. Regarding proximal and distal positions referenced herein, the proximal end of a prosthesis (e.g., stent-graft) is the end closest to the heart (by way of blood flow) whereas the distal end is the end furthest away from the heart during deployment. In contrast, the distal end of a catheter is usually identified as the end that is farthest from the operator, while the proximal end of the catheter is the end nearest the operator. Once the catheter is positioned for deployment of the stent-graft at the target site, the inner tube is held stationary and the outer tube (sheath) withdrawn so that the stent-graft is gradually exposed and expands. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Although the endovascular approach is much less invasive, and usually requires less recovery time and involves less risk of complication as compared to open surgery, there can be concerns with alignment of asymmetric features of various prostheses in relatively complex applications such as one involving branch vessels. Branch vessel techniques have involved the delivery of a main device (e.g., a graft or stent-graft) and then a secondary device (e.g., a branch graft or branch stent-graft) through a fenestration or side opening in the main device and into a branch vessel.

The procedure becomes more complicated when more than one branch vessel is treated. One example is when an aortic abdominal aneurysm is to be treated and its proximal neck is diseased or damaged to the extent that it cannot support a reliable connection with a prosthesis. In this case, grafts or stent-grafts have been provided with fenestrations or openings formed in their side wall below a proximal portion thereof. The fenestrations or openings are to be aligned with the renal arteries and the proximal portion is secured to the aortic wall above the renal arteries.

To ensure alignment of the prostheses fenestrations and branch vessels, some current techniques involve placing guidewires through each fenestration and branch vessel (e.g., artery) prior to releasing the main device or prosthesis. This involves manipulation of multiple wires in the aorta at the same time, while the delivery system and stent-graft are still in the aorta. In addition, an angiographic catheter, which may have been used to provide detection of the branch vessels and preliminary prosthesis positioning, may still be in the aorta. Not only is there risk of entanglement of these components, the openings in an off the shelf prosthesis with preformed fenestrations may not properly align with the branch vessels due to differences in anatomy from one patient to another. Prostheses having preformed custom located fenestrations or openings based on a patient's CAT scans also are not free from risk. A custom designed prosthesis is constructed based on a surgeon's interpretation of the scan and still may not result in the desired anatomical fit. Further, relatively stiff catheters are used to deliver grafts and stent-grafts and these catheters can apply force to tortuous vessel walls to reshape the vessel (e.g., artery) in which they are introduced. When the vessel is reshaped, even a custom designed prosthesis may not properly align with the branch vessels.

Generally speaking, physicians often use fluoroscopic imaging techniques for stent-graft positioning and deployment. Fluoroscopy allows one to observe real-time X-ray images of the target organs and/or aorta. This approach requires one to administer a radiopaque substance, which generally is referred to as a contrast medium, agent or dye, into the patient so that it reaches the area to be visualized (e.g., the renal arteries). A catheter can be introduced through the femoral artery in the groin of the patient and endovascularly advanced to the vicinity of the renals. The fluoroscopic images of the transient contrast agent in the blood, which can be still images or real-time motion images, allow two dimensional visualization of the location of the renals.

The use of X-rays, however, requires that the potential risks from a procedure be carefully balanced with the benefits of the procedure to the patient. While physicians always try to use low dose rates during fluoroscopy, the duration of a procedure may be such that it results in a relatively high absorbed dose to the patient. Patients who cannot tolerate contrast enhanced imaging or physicians who must or wish to reduce radiation exposure need an alternative approach for defining the vessel configuration and branch vessel location.

Accordingly, there remains a need to develop and/or improve endovascular prosthesis deployment apparatus and methods.

### SUMMARY OF THE INVENTION

The present invention involves improvements in prosthesis deployment apparatus and methods.

In one embodiment according to the invention, tubular prosthesis apparatus comprises a tubular member (e.g., stent-graft) having a proximal end portion and a distal end portion and being adapted for endovascular delivery to a site in a patient; and an electromagnetic localization marker (e.g., a sensing coil) and signal lead, the signal lead being electrically coupled to the electromagnetic localization marker and releasably coupled to the proximal end portion of the tubular member.

Other features, advantages, and embodiments according to the invention will be apparent to those skilled in the art from the following description and accompanying drawings.

### BRIEF DESCRIPTION OF THE DRAWINGS

FIG. 1 diagrammatically illustrates one embodiment of a prosthesis delivery system in accordance with the invention.

FIG. 2A illustrates one embodiment of prosthesis delivery system of FIG. 1 in a loaded state.

FIG. 2B illustrates the prosthesis delivery system of FIG. 2A in a partially deployed state.

FIG. 2C illustrates the prosthesis delivery system of FIG. 2B with the prosthesis proximal end deployed.

FIG. 3A diagrammatically illustrates one embodiment of a removable lead system for a prosthesis localization device according to the invention.

FIG. 3B is a view of the prosthesis-localization device of FIG. 3A taken along line FIG. 3B-3B.

FIG. 3C diagrammatically illustrates withdrawal of the localization devices and accompanying leads shown in FIG. 3 from the prosthesis.

FIG. 4A diagrammatically illustrates another embodiment of a removable lead system for a prosthesis localization device according to the invention.

FIG. 4B diagrammatically illustrates one of the removable of the leads of FIG. 4A with a sacrificial link.

FIG. 4C diagrammatically illustrates the lead of FIG. 4B with the sacrificial link removed.

FIG. 5A diagrammatically illustrates a marker localization system coupled to the prosthesis delivery system of FIG. 1 with an optional circuit for electrolysis of the sacrificial link shown in FIG. 4B in an off position.

FIG. 5B illustrates the system of claim 5A switched on for electrolysis of the sacrificial link.

### DETAILED DESCRIPTION

The following description will be made with reference to the drawings where when referring to the various figures, it should be understood that like numerals or characters indicate like elements.

Regarding proximal and distal positions, the proximal end of the prosthesis (e.g., stent-graft) is the end closest to the heart (by way of blood flow) whereas the distal end is the end farthest away from the heart during deployment. In contrast, the distal end of the catheter is usually identified as the end that is farthest from the operator, while the proximal end of the catheter is the end nearest the operator. Therefore, the prosthesis (e.g., stent-graft) and delivery system proximal and distal descriptions may be consistent or opposite to one another depending on prosthesis (e.g., stent-graft) location in relation to the catheter delivery path.

Placing electromagnetic sensing coils or markers with signal transmitting leads on a prosthesis can facilitate real-time data acquisition of the position of the coils in three-dimensional space, which can be helpful with in vivo manipulation of the instruments. Embodiments of the invention facilitate removal of marker localization leads after deployment of prosthesis (e.g., a stent-graft) having electromagnetic localization markers with signal transmitting leads coupled thereto.

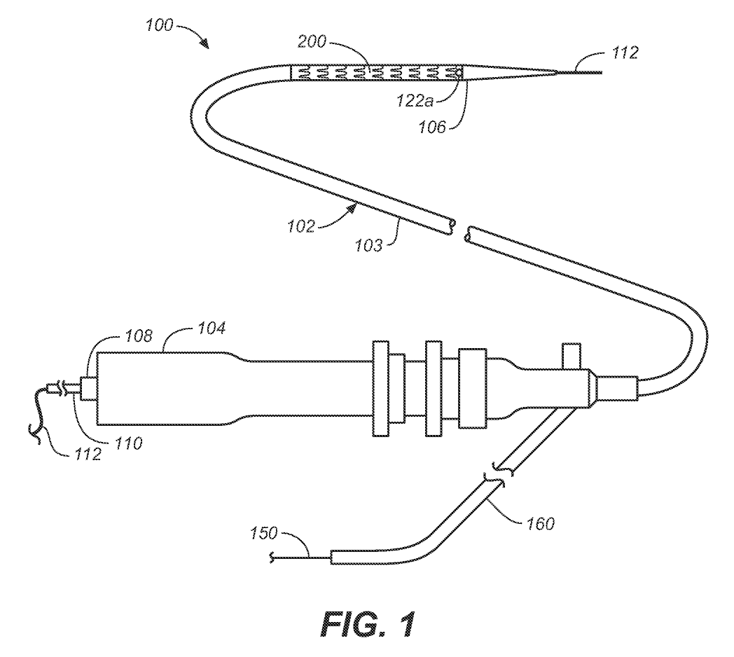

Referring to FIG. 1, one embodiment of a prosthesis delivery system according to the invention is shown and generally designated with reference numeral 100. Prosthesis delivery system 100 comprises catheter 102, control handle 104, flexible tapered tip member (or obturator or regular blunt tip or otherwise) 106, which can form a portion of the distal end of the catheter. Handle 104 includes an inlet 108, through which central guidewire lumen 110 enters the handle and extends to flexible tapered tip member 106, which has an axial bore for slidably receiving guidewire 112. Tapered tip member 106 is placed at the distal end of catheter sheath 103 and handle 104 is affixed to the proximal end of catheter sheath 103. A guidewire 112 can be slidably disposed in guidewire lumen 110 and catheter 112 tracked thereover. When the prosthesis to be delivered is a self-expanding graft or stent-graft (such as stent-graft 200), it generally is radially compressed or folded and placed in the distal end portion of the delivery catheter and allowed to expand upon deployment from the catheter at the target site as will be described in detail below. Stent-graft 200 can include a plurality of undulating stent elements to support the tubular graft material as is known in the art.

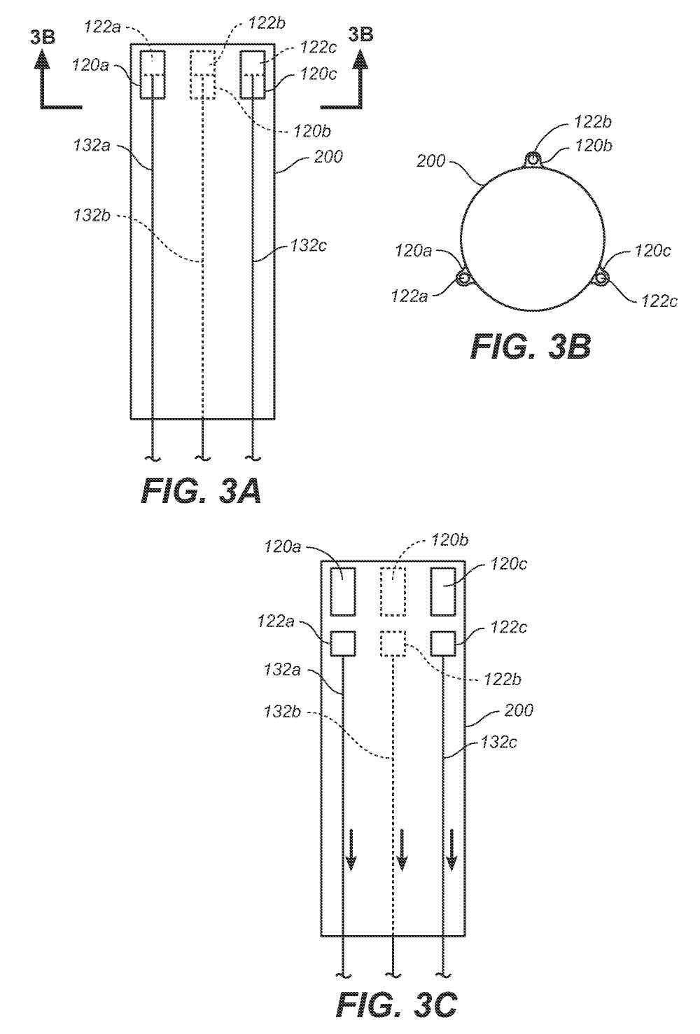

Referring to FIG. 1, one or more markers 122a, 122b, 122c . . . 122n are attached to or integrally formed in a proximal portion of prosthesis 200 with their signal leads releasably coupled to the prosthesis. The markers can be equidistantly spaced from one another in a circumferential direction or spaced otherwise depending on the nature of the image desired. In the example shown in FIG. 3A, three equidistantly spaced localization markers are depicted.

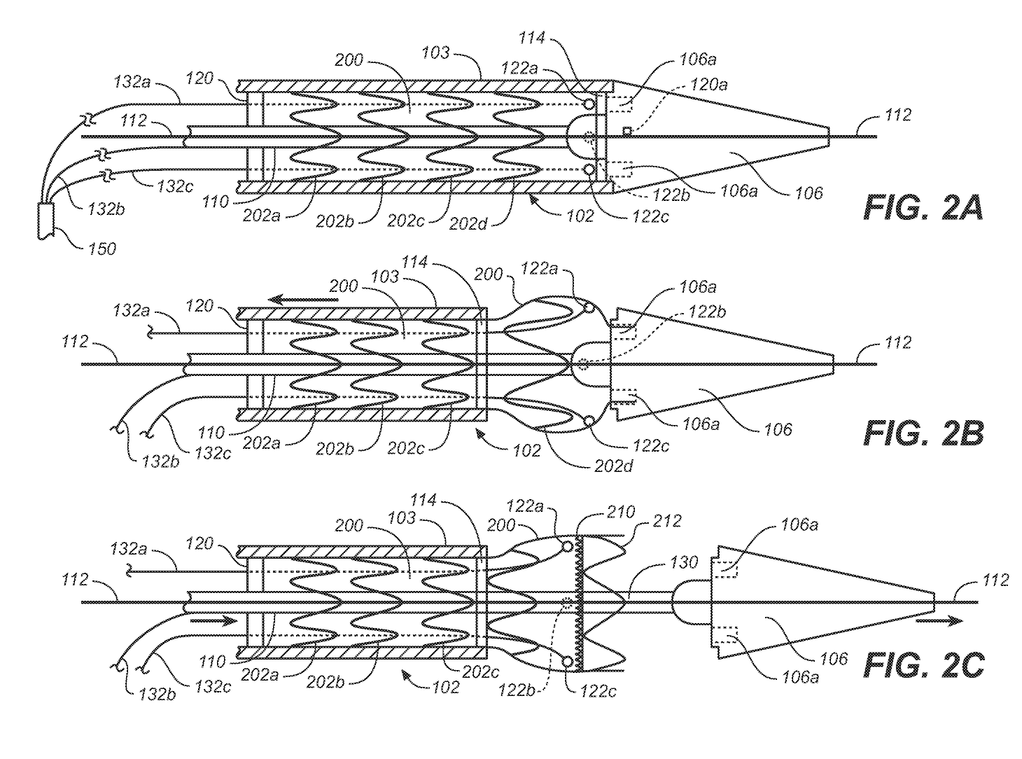

Referring to FIGS. 2A-C, one delivery catheter system for delivering prosthesis of the invention is shown in a pre-deployment loaded state FIG. 2A and two partial deployment states (FIGS. 2B & 2C). Delivery catheter 102 includes catheter sheath 103, which can be referred to as an outer tube, and inner guidewire tube 110. Sheath 103 and guidewire tube 110 are coaxial and arranged for relative axial movement therebetween. The prosthesis (e.g., stent-graft 200) is positioned within the distal end of outer tube 103 and in front of pusher member or stop 120, which is concentric with and secured to inner guidewire tube 110 and can have a disk or ring shaped configuration with a central access bore to provide access for guidewire tube 110. In the example where prosthesis 200 comprises a stent-graft as shown in the illustrative embodiment, the stent graft comprises a tubular graft member and a plurality of annular undulated stent elements, such as stent elements 202a,b,c,d, to provide structural support to the graft as is known in the art. An undulating bear spring element 212 also can be sutured or otherwise attached to the proximal end of the prosthesis and/or an annular undulating wire 210 having an undulating configuration secured to the proximal end of the prosthesis to provide radial strength as well. Bare spring 212 has a radially outward bias so that when it is released from a radially collapsed or restrained state it expands outwardly to secure the proximal portion of the prosthesis to the target passageway wall. Another undulating wire 210 can be attached to the prosthesis distal end in addition to or in place of a proximal undulating wire 210. More specifically, a support spring 210 can be provided at one or both ends of the prosthesis. The stent and support elements can be positioned on the interior and/or exterior of the graft member and secured thereto by suturing or other conventional means. The tubular graft material which the stent structures support can be any suitable material such as Dacron® or expanded polytetrafluoroethylene (ePTFE).

A radiopaque ring 114 can be provided on the inside of the distal end portion of sheath 103 in overlapping relation to the tapered tip (FIG. 2A) to assist with imaging the distal end of sheath 103 using fluoroscopic techniques. Alternatively, radiopaque ring 114 can be provided on the proximal end of the tapered tip.

Once the catheter is positioned for deployment of the prosthesis at the desired site, the inner member or guidewire lumen 110 with stop 120 are held stationary and the outer tube or sheath 103 withdrawn so that sheath 103 is displaced from tapered tip 106 and the stent-graft gradually exposed and allowed to expand. Stop 120 therefore is sized to engage the distal end of the stent-graft as the stent-graft is deployed. The proximal ends of the sheath 103, inner tube or guidewire lumen 110, and guidewire 112 are coupled to and manipulated by handle 104. Tapered tip 106 optionally can include a stent graft proximal end holding mechanism to receive and hold the proximal end of the stent-graft so that the operator can allow expansion of the stent-graft proximal end during the last phase of its deployment. In this regard, Alternatively, any of the stent-graft deployment systems described in U.S. Patent Application Publication No. 2004/0093063, which published on May 13, 2004 to Wright et al. and is entitled Controlled Deployment Delivery System, the disclosure of which is hereby incorporated herein in its entirety by reference thereto, can be incorporated into stent-graft delivery system 100.

Referring to FIG. 2B, the prosthesis delivery system is shown with catheter sheath 103 partially pulled back and a portion of the prosthesis partially expanded. In this partially retracted position, the proximal end of the prosthesis is constrained allowing the prosthesis to be repositioned (e.g., longitudinally or rotationally moved) if desired before release of the proximal end of the prosthesis. The surgeon can determine if prosthesis repositioning is desired based on monitored movement of the prosthesis during deployment as will be described in more detail below. Referring to FIG. 2C, the catheter sheath is held stationary and guide lumen 110, which is fixedly secured to tapered tip 106, is advanced to separate tapered tip 106 from catheter sheath 103 and release the proximal end of prosthesis 200.

Referring to FIG. 1, the prosthesis has electromagnetic localization markers 122a, 122b, 122c . . . 122n each having a separate lead 132a, 132b, 132c . . . 132n, which extend between the prosthesis and catheter sheath or between the prosthesis and guidewire tube 110 and are bundled in cable 150. Cable 150 extends into sleeve 160 to a signal measuring and processing circuit as will be described in more detail below. The positions of the electromagnetic localization markers can be located in vivo by determining the positions of the coils relative to a plurality of magnetic field sources of known location. See U.S. Pat. No. 5,913,820 to Bladen, et al. and entitled Position Location System, the disclosure of which is hereby incorporated herein in its entirety by reference thereto, which describes a system for determining the position of electromagnetic coil markers.

Generally speaking, pre-specified electromagnetic fields are projected to the portion of the anatomical structure of interest (e.g., that portion that includes all prospective locations of the electromagnetic localization markers or sensing coils in a manner and sufficient to induce voltage signals in the marker coils. Electrical measurements of the voltage signals are made to compute the angular orientation and positional coordinates of the electromagnetic markers or sensing coil and hence the location of the vasculature and/or devices of interest. An example of sensing coils for determining the location of a catheter or endoscopic probe inserted into a selected body cavity of a patient undergoing surgery in response to pre-specified electromagnetic fields is disclosed in U.S. Pat. No. 5,592,939 to Martinelli and entitled System And Method For Navigating A multiple Electrode Catheter, the disclosure of which is hereby incorporated herein in its entirety by reference thereto. Also see U.S. Pat. No. 5,913,820 to Bladen, et al. (supra), which describes methods and apparatus for locating the position in three dimensions of a sensor comprising a sensing coil by generating magnetic fields which are detected at the sensor.

The Stealth Station®AXIEM™ electromagnetic technology is another example of an electromagnetic guided platform and is available from Medtronic, Inc. (Minneapolis, Minn.). Further, the Magellan electromagnetic navigation system (Biosense Webster, Tirat HaCarmel, Israel) is yet another electromagnetic platform.

Referring to FIGS. 3A-C, one embodiment of a prosthesis with localization markers and their signal leads releasably coupled to the proximal end of the prosthesis is shown. Prosthesis 200 has a plurality of pockets 120a, 120b, and 120c formed therein for releasably holding localization markers 122a, 122b, and 122. Each pocket can be made from a piece of graft material that is sutured or otherwise secured to the proximal end portion of the prosthesis such that an opening faces distally of the prosthesis (FIG. 3B) for receiving and releasing a respective marker (FIG. 3C). Although three localization markers 122a, 122b, and 122c are shown, other multiples of markers can be used depending on, for example, the desired nature of the image to be displayed. Electrically conductive wire leads 132a, 132b, and 132c extend from respective markers 122a, 122b, and 122c to bundle cable sheath 150 as described above and can comprise for example stainless steel coated copper, stainless steel or other coated or bare wire which has a low rate of reaction to the human body environment in which it is to be used.

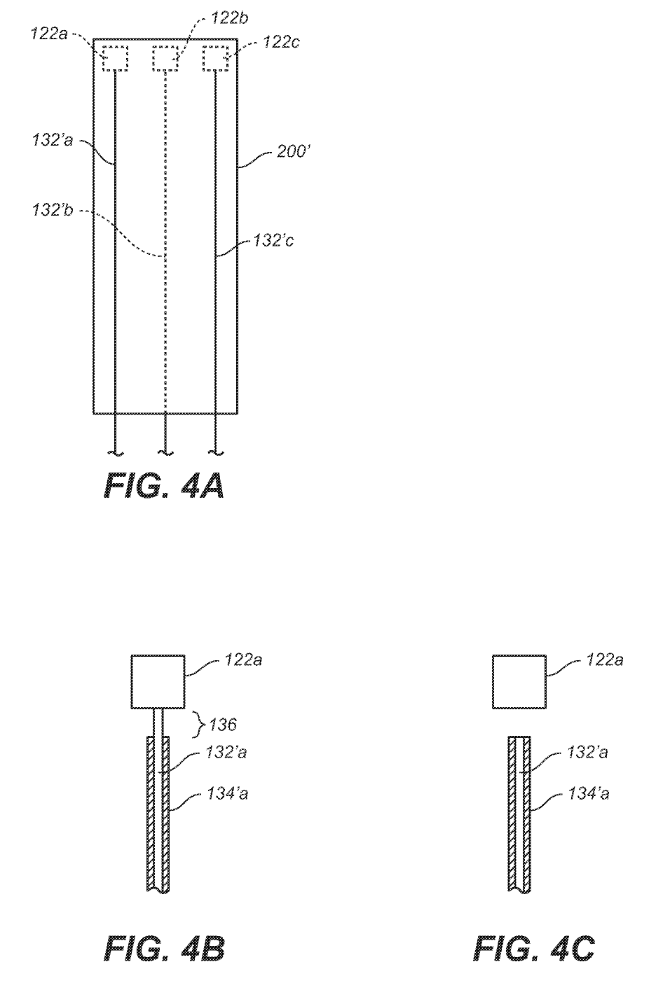

Referring to FIGS. 4A-C, another prosthesis 200′, which can be a stent-graft, has an alternative lead release mechanism. In this embodiment, leads 132′a, 132,b′, and 132′c are electrically coupled to electromagnetic markers or sensing coils 122a, 122b, and 122c through an electrolytically disintegrateable link. The electromagnetic markers can be sutured to the prosthesis or secured thereto with other known suitable means.

Referring to FIG. 4B an enlarged view an electrolytically disintegratable link, which is generally designated with reference numeral 136 is shown. Although link 136 is shown with respect to electromagnetic localization marker or sensing coil 122a, the same link construction can be provided in the leads to the other markers or coils.

According to one embodiment, link 136 is a bare portion of lead 132′a, which otherwise is insulated with insulation coating or jacket 134′a. Link 136 electrically couples lead 132′a to electromagnetic sensing coil 122, which is otherwise insulated and can be encased in an insulating biocompatible housing. Link 136, which can be an extension or portion of lead 132′, also can be an extension of the encased marker coil, which extends through the encasement. Alternatively, link 136 can be welded, soldered, brazed or otherwise fixed to a conductive member that extends from the marker coil through the marker coil housing to allow voltage signals from the marker coil to pass through the sacrificial link and lead 132′a.

Since electrolytically disintegratable link 136 is bare, it is relatively more susceptible to electrolysis in an ionic solution such as blood or most other bodily fluids than insulation coating or jacket 134′a and the insulation or housing surrounding the electromagnetic sensing coil.

In one variation, link 136 may be tapered or otherwise modified, or coated with an insulative polymer and scored, such as described in U.S. Pat. No. 5,624,449 to Pham et al. and entitled Electrolytically Severable Joint For Endovascular Embolic Devices, the disclosure of which is hereby incorporated herein in its entirety by reference thereto, to limit the area of electrolytic disintegration to a more discrete region or point.

Other examples of electrolytically disintegratable links are described in U.S. Pat. No. 6,623,493 to Wallace, et al. and entitled Vaso-Occlusive member Assembly With Multiple Detaching Points, U.S. Pat. No. 6,425,914 to Wallace, et al. and entitled Fast-Detaching Electrically Insulated Implant, U.S. Pat. No. 6,589,230, to Gia, et al. and entitled System For Detaching An Occlusive Device Within A Mammalian Body Using A Solderless, Electrolytically Severable Joint, U.S. Pat. No. 6,123,714 to Gia, et al. and entitled System For Detaching An Occlusive Device Within A Body Using A Solderless, Electrolytically Severable Joint, U.S. Pat. No. 5,891,128 to Gia, et al. and entitled Solderless Electrolytically Severable joint For Detachable Devices Placed Within The Mammalian Body, U.S. Pat. No. 5,669,905 to Scheldrup, et al. and entitled Endovascular Embolic Device Detachment Detection Method And Apparatus, and U.S. Patent Application Publication No. 2003/0130689 to Wallace, et al., which is entitled Vaso-Occlusive member Assembly With Multiple Detaching Points and published on Jul. 10, 2003, all of the disclosures of which are hereby incorporated herein in their entirety by reference thereto.

Suitable electrically insulative materials are a biocompatible, electrically insulative material such as polyfluorocarbons (e.g. TEFLON), polyethylene terepthalate, polypropylene, polyurethane, polyimides, polyvinylchloride, and silicone polymers. In addition to these polymers, another suitable material is generically known as parylene. There are a variety of polymers (e.g., polyxylylene) based on para-xylylene. These polymers are typically placed onto a substrate by vapor phase polymerization of the monomer. Parylene N coatings are produced by vaporization of a di(P-xylylene) dimer, pyrolization, and condensation of the vapor to produce a polymer that is maintained at a comparatively lower temperature. In addition to parylene-N, parylene-C is derived from di(monochloro-P-xylylene) and Parylene-D is derived from di(dichloro-P-xylylene). There are a variety of known ways to apply parylene to substrates. Their use in surgical devices has been shown, for instance, in U.S. Pat. No. 5,380,320 (Morris), U.S. Pat. No. 5,174,295 (Christian et al.), U.S. Pat. No. 5,067,491 (Taylor et al.), and the like.

The insulation can be applied using well known techniques. In one example, a respective lead is dipped in a molten or substantially softened polymer material and insulation in the region of link 136 removed. Other approaches known in the art include, but are not limited to shrink-wrapping and line-of-sight deposition in the form of a suspension or latex.

Typically the thickness of the lead insulation can range from about 0.002 inch to 0.040 inch, and can be in the range of about 0.003 inch to 0.0010 inch. The thickness of the localization marker coil casing or housing depends on the desired thermal, electrical and/or mechanical properties of thereof.

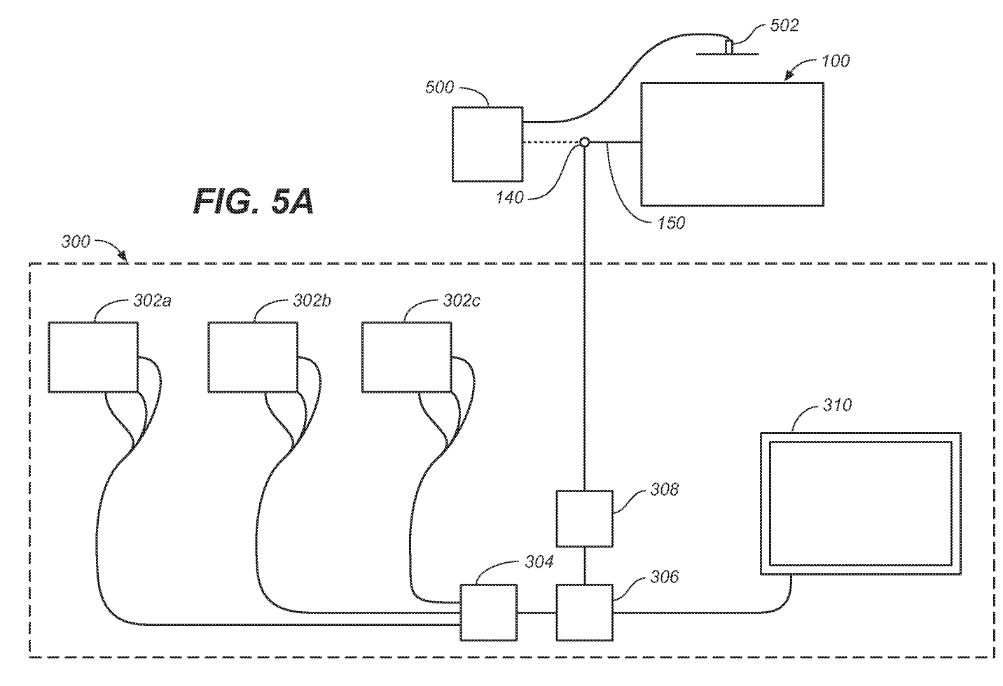

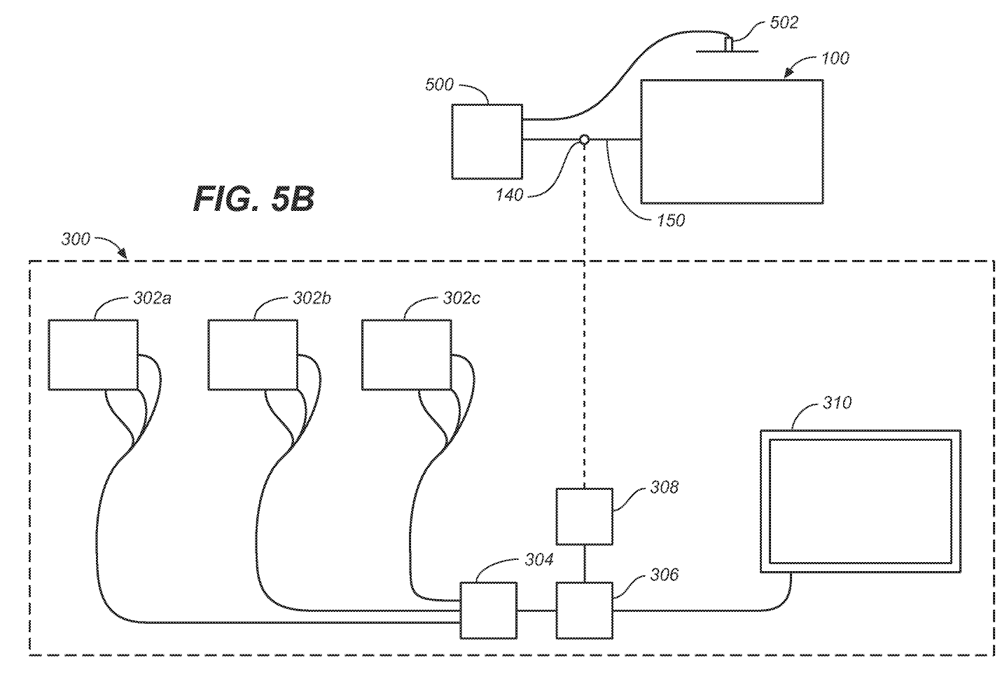

Referring to FIGS. 5A-B, one localization system 300 for providing the position or orientation of a target (e.g., the electromagnetic localization markers) in three dimensions is shown in combination with optional system 500 for activating electrolysis of optional sacrificial link 136 described above. A conventional switching circuit 400 provides a switching mechanism to electrically couple either system 300 or system 500 to the localization markers. In FIG. 5A, system 300 is shown electrically coupled to the localization markers and system 500 decoupled therefrom. In FIG. 5B, system 300 is shown electrically decoupled from the localization markers and system 500 coupled thereto.

Systems 300 and 500 can comprise use known circuits used for the purpose described above. See, for example, U.S. Pat. No. 5,913,820 to Bladen et al. (supra) regarding localization system 300 and U.S. Pat. No. 5,669,905 to Scheldrup, et al. (supra) regarding electrolytic disintegration system 500.

Field generating and signal processing circuit 300 generates magnetic fields about the electromagnetic localization markers or sensors and processes the voltage signals that the markers or sensors generate in response to the generated magnetic fields. Such a field generating and signal processing circuit is described in U.S. Pat. No. 5,913,820 to Bladen et al. (supra). Circuit 300 generally includes three electromagnetic field (EMF) generators 302a, 302b, and 302c, amplifier 304, controller 306, measurement unit 308, and display device 310. Each field generator comprises three electrically separate coils of wire (generating coils). The nine generating coils are separately electrically connected to amplifier 304, which drives each coil individually and sequentially in response to the direction of controller 306.

Once the quasi-static field from a particular generating coil is established, measurement unit 308 measures the value of the voltage that the field induces in each sensing coil. This data is processed and passed to controller 306, which stores the value and then instructs amplifier 304 to stop driving the present generating coil and to start driving the next generating coil. When all generating coils have been driven, or energized, and the corresponding nine voltages induced into each sensing coil have been measured and stored, controller 306 calculates the location and orientation of each sensor relative to the field generators and displays this on a display device 310. This calculation can be carried out while the subsequent set of nine measurements is being taken. Thus, by sequentially driving each of the nine generating coils, arranged in three groups of three mutually orthogonal coils, the location and orientation of each sensing coil can be determined.

The sensor and generating coil specifications, as well as the processing steps are within the skill of one of ordinary skill of the art. An example of coil specifications and general processing steps that can be used are disclosed in U.S. Pat. No. 5,913,820 to Bladen, et al.(supra). Also see the coils in U.S. Pat. No. 5,592,939 to Martinelli (supra) and U.S. Pat. No. 6,889,833 to Seiler, et al. and entitled Packaged Systems For Implanting Markers In A Patient And Methods For Manufacturing And Using Such Systems, the disclosure of which is hereby incorporated herein in its entirety by reference thereto.

The following is one exemplary procedure after which removal of the voltage signal leads is performed according to the embodiments of the invention described above. For the purposes of the example, the procedures involve the endovascular delivery and deployment of an AAA bifurcated stent-graft in a patient's aorta below the renal arteries.

Prior to the surgical procedure, the patient is scanned using either a CT or MRI scanner to generate a three-dimensional model of the vasculature to be tracked. Therefore, the aorta and renal arteries can be scanned and images taken thereof to create a three-dimensional pre-procedural data set for that vasculature and create a virtual model upon which real-time data can be overlaid. The virtual model is input into the software that consolidates all of the navigation information and displays information that the user sees.

The magnetic field generators are positioned on the operating table to facilitate triangulation of the exact position of sensor in three-dimensional space using xyz coordinates.

The patient is prepared for surgery and a cut is made down to a femoral artery and a guidewire inserted. A device which measures the position of an anatomical reference point of the patient in the magnetic field is placed as a reference, either external to the patient or intravascularly. Examples of external markers are fiducial markers on the patient's skin over vertebra or on skin over the iliac crest. Examples of internal markers are a coil placed in the wall of the renals arteries of other branches of the aorta. A contrast agent catheter is delivered through the femoral artery and the vasculature perfused with contrast and a fluoroscopic image up to the renal arteries taken. The processor orients or registers the three-dimensional virtual image to the fluoroscopic X-ray image. Such registration is known in the art.

The operator tracks catheter 102 over guidewire 112 toward the aneurysm and renal arteries. The position of the distal end of catheter sheath 103 is monitored virtually based on the signals received from the localization markers on the stent-graft and the known catheter dimensions and position of the markers relative to distal end of the catheter sheath 103, all of which were entered into the processor, to indirectly provide the position of the proximal end of stent-graft 200.

The position of the localization markers and/or the catheter sheath distal end as they move toward the lower renal artery can be displayed on a monitor. Alternatively, the localization markers and/or the stent-graft proximal end can be displayed.

In the vicinity of the target location (e.g., the lower renal artery), which the operator can estimate based on the three-dimensional model and the localization marker positions, the operator can use fluoroscopy to position the stent-graft at the desired location below the lower renal ostia. Fluoroscopy also can be helpful in the positioning step when the aorta is very tortuous and the catheter significantly changed the aorta's configuration (the configuration of the virtual model) during advancement therethrough.

Once the stent-graft is in the desired position, the operator holds guidewire tube 110 and pusher disk 120 stationary, and retracts or pulls back catheter sheath 103. As the sheath is pulled back, the positions of the localization markers or sensors are monitored to determine if movement beyond a threshold value occurs. A virtual image of the sensors and stent-graft can be displayed on the monitor so that the surgeon can monitor the sensors qualitatively, either individually or in coordination with 2D or 3D images generated pre-operatively or intra-operatively. Alternatively, the processor can display a warning when the sensors move more than a threshold value (e.g., more than 2 mm) in the direction of blood flow or in a direction generally parallel to the central axis of the proximal end portion of the stent-graft. In a further alternative, the distance between the base of the ostia and the sensors, measured in the direction of blood flow or along the aortic wall in a distal direction (away form the heart), is displayed to provide a quantitative value of stent-graft movement in that direction. A non-symmetric marker array pattern will provide a precise incremental image of any rotation or other translation of the prosthesis. In contrast to a symmetrical array whose image would repeat and might be indistinguishable if it were rotated, by exactly one incremental mark. In yet a further alternative, the annular proximal end of the stent-graft can monitored based in stent-graft dimensional data and the position of the sensor relative to the proximal end of the stent-graft, which can be readily input into the processor.

If the position of the stent-graft proximal portion changes more than a desired amount, the operator repositions the stent-graft. The operator simply repositions the stent-graft or can involve moving the sheath over the expanded portion of the stent-graft to allow the stent-graft to be more readily repositioned. The stent-graft is then deployed again while monitoring movement during deployment. Once the prosthesis is deployed, the voltage signal leads and catheters are removed.

According to the embodiment illustrated in FIGS. 3A-C, leads 132a, 132b, and 132c are pulled in the direction of the arrows shown in FIG. 3C, which withdraws localization markers 122a, 122b, 122a from pockets 120a, 120b, and 120c and allows removal of the leads and markers from the patient.

According to the embodiment illustrated in FIGS. 4A-C, switching circuit 400 is set to uncouple localization system or circuit 300 from the localization markers and to couple lead detachment system or circuit 500, which includes a power supply and a processor, to remove the sacrificial links. Then a positive electric current of approximately 0.01 to 2 milliamps at 0.1 to 6 volts is applied to the localization marker leads. Typically, the negative pole of the power supply is placed in electrical contact with the patient's skin as diagrammatically illustrated with electrode 502 in FIGS. 5A and 5B. The sacrificial links undergo electrolytic disintegration to cause a break between the portion of voltage signal lead proximal thereto and the localization marker. This may occur within 0.5 to 10 minutes.

Once the breaks or complete disintegration of the sacrificial links have occurred the leads and catheters are removed. For example, once the link has disintegrated the circuit will be cut and the resistance will rise to infinity or to the resistance value associated with the tissue surrounding the target sensor.

Any feature described in any one embodiment described herein can be combined with any other feature of any of the other embodiments whether preferred or not. Variations and modifications of the devices and methods disclosed herein will be readily apparent to persons skilled in the art.

What is claimed is:

1. Tubular prosthesis apparatus comprising:
a tubular member having a proximal end portion and a distal end portion and being adapted for endovascular delivery to a site in a patient; and
an electromagnetic localization marker and signal lead, said signal lead being electrically coupled to said electromagnetic localization marker and releasably coupled to said proximal end portion of said tubular member.

2. The prosthesis apparatus of claim 1 wherein said tubular member has a pocket and said electromagnetic localization marker is releasably retained in said pocket.

3. The prosthesis apparatus of claim 2 wherein said tubular member is a stent-graft.

4. The prosthesis apparatus of claim 1 wherein said lead has an electrolytically disintegratable link adjacent to said electromagnetic localization marker.

5. The prosthesis apparatus of claim 4 further including insulation surrounding said lead proximal to said link and insulation surrounding said electromagnetic localization marker.

6. The prosthesis apparatus of claim 5 wherein said prosthesis is a stent-graft.

7. The prosthesis apparatus of claim 1 wherein said prosthesis is a stent-graft.
